(19) United States
(12) Patent Application Publication
(10) Pub. No.: US 2010/0030006 A1
(43) Pub. Date: Feb. 4, 2010

(54) MEMBRANE-MEDIATED EXTRACTION OF OLEFINS

(75) Inventor: Robert C. Schucker, The Woodlands, TX (US)

Correspondence Address: WINSTEAD PC, P.O. BOX 50784, DALLAS, TX, 75201, US

(73) Assignee: The Ionics Corporation, The Woodlands, TX (US)

(21) Appl. No.: 12/481,190

(22) Filed: Jun. 9, 2009

Related U.S. Application Data
(60) Provisional application No. 61/060,052, filed on Jun. 9, 2008.
(60) Provisional application No. 61/060,045, filed on Jun. 9, 2008.
(60) Provisional application No. 61/060,056, filed on Jun. 9, 2008.
(60) Provisional application No. 61/060,044, filed on Jun. 9, 2008.

(51) Int. Cl. C07C 7/144

(52) U.S. Cl. 585/818

(57) ABSTRACT

The present disclosure provides processes for the recovery of olefins from a mixture. Such processes include: (1) passing the mixture to a first surface of a porous membrane, wherein the porous membrane is in a first contactor; (2) passing a complexing composition to a second surface of the porous membrane in the first contactor, wherein the complexing composition includes a metal ion that is capable of reversibly binding the olefins; (3) extracting at least a portion of the olefins from the mixture; wherein extracting includes binding the olefins to the metal ion of the complexing composition; and wherein extracting produces a mixture depleted in olefins and a complexing composition enriched in olefins; and (4) inducing a release of the olefins from the complexing composition enriched in olefins. In some embodiments, the processes of the present disclosure can further include passing the complexing composition enriched in olefins to a second contactor; wherein the induction step occurs in the second contactor. In other embodiments, the processes of the present disclosure can further include passing the released olefins from the induction step to a third contactor. In additional embodiments, the processes of the present disclosure can also include recycling the complexing composition after the olefins are released in the induction step.

### CROSS-REFERENCE TO RELATED APPLICATIONS

This application claims priority to U.S. Provisional Patent Application Nos. 61/060,052, 61/060,045, 61/060,056, and 61/060,044, all filed on Jun. 9, 2008, and all incorporated herein by reference in their entirety.

### STATEMENT REGARDING FEDERALLY SPONSORED RESEARCH

The United States Government has rights in this invention pursuant to Contract No. DE-FG02-05ER84262 between the United States Department of Energy and Translonics Corporation.

### FIELD OF THE INVENTION

The present invention relates to membrane-based processes to separate olefins from mixtures, such as mixed olefin/paraffin streams in a gaseous or liquid state.

### BACKGROUND

Many olefins, such as ethylene and propylene, can be produced by various processes operated by the chemical and refining industries. One of such processes is steam cracking of feeds such as ethane, propane, butane, naphtha or gas oil. A preferred feed stock for such a process is the natural gas liquids (NGL) stream because of high yields of desired products. Another process involves the recovery of light ends from fluid catalytic cracking. In both such cases, however, the products of the conversion reactors are mixtures of chemical species that generally require additional separation and purification steps.

Traditionally, additional separation and purification steps of olefins have been done by distillation. For instance, the separation of ethylene from ethane or propylene from propane by distillation has been typically accomplished under cryogenic conditions at elevated pressures due to the low boiling points of these liquids. Cryogenic distillation, however, is extremely energy intensive, resulting in substantial costs to separate olefins from paraffins. For instance, it has been estimated that such separations may account for 6.3% (about 0.15 quadrillion BTUs) of the energy used by the chemical and petrochemical industries.

Furthermore, there are numerous examples of mixed liquid olefin/paraffin streams that cannot be effectively separated by distillation because of similarities in boiling points. One example of such a stream is a byproduct of the synthesis of ethylene-1-octene copolymer, which includes a mixture of a paraffinic solvent and more than a dozen C₈ olefins.

One potential lower energy olefin/paraffin separation process is based on complexation of metals with the olefins. In particular, because of the π electrons in their double bonds, olefins can complex reversibly with certain metals or metal ions, particularly with transition metal ions, such as, for example, Ag⁺ and Cu⁺. Furthermore, many such complexation processes utilize membranes, which contain the complexing agent. Such membranes are typically referred to as facilitated transport membranes, since the complexing agents enhance or facilitate the diffusion of olefins relative to non-olefins across the membrane. Facilitated transport membranes in conventional olefin separations tend to be relatively thick, thus providing a longer distance over which diffusion must occur. The longer diffusion distance in turn may result in a lower flux and a lower olefin recovery rate.

Therefore, there is a current need for improved membrane-mediated olefin separation methods that are more effective in recovering olefins. There is also a need for olefin separation methods that are less energy intensive than those presently used in the art.

### SUMMARY

In some embodiments, the present disclosure provides processes for the recovery of olefins from a mixture. Such processes generally include: (1) passing the mixture to a first surface of a porous membrane in a first contactor; (2) passing a complexing composition with a metal ion capable of reversibly binding the olefins to a second surface of the porous membrane in the first contactor; (3) extracting at least a portion of the olefins from the mixture by binding the olefins to a metal ion of the complexing composition; and (4) inducing a release of the olefins from the complexing composition.

In additional embodiments, the induction step occurs in a second contactor. In such embodiments, the processes further include passing the olefin-enriched complexing composition to a second contactor for the induction step to occur. In further embodiments, the processes of the present disclosure can further include passing the released olefins from the induction step to a third contactor. In additional embodiments, the processes of the present disclosure can also include recycling the complexing composition after the release of olefins in the induction step.

### BRIEF DESCRIPTION OF THE DRAWINGS

For a more complete understanding of the present disclosure, and the advantages thereof, reference is now made to the following descriptions to be taken in conjunction with the accompanying drawings describing specific embodiments of the disclosure, wherein:

FIG. 1 provides an overall schematic of an illustrative olefin recovery process in accordance with some embodiments of the present disclosure.

FIG. 2A is an illustrative depiction of a first mode of operation of a first contactor for recovering olefins through the processes of the present disclosure.

FIG. 2B is an illustrative depiction of a second mode of operation of a first contactor for recovering olefins through the processes of the present disclosure.

FIG. 3 is an illustrative depiction of a second contactor suitable for use with the olefin recovery processes of the present disclosure. In this embodiment, the second contactor is operated under vacuum.

FIG. 4 provides the structures of several bidentate and tridentate ligands as non-limiting examples of ligands that can be used with the complexing compositions of the present disclosure.

### DETAILED DESCRIPTION

In the following description, certain details are set forth such as specific quantities, concentrations, sizes, etc. so as to provide a thorough understanding of the various embodiments disclosed herein. However, it will be apparent to those skilled in the art that the present disclosure may be practiced without such specific details. In many cases, details concerning such considerations and the like have been omitted inasmuch as such details are not necessary to obtain a complete understanding of the present disclosure and are within the skills of persons of ordinary skill in the relevant art.

Likewise, referring to the drawings in general, it will be understood that the illustrations are for the purpose of describing particular embodiments of the disclosure and are not intended to be limiting thereto. Furthermore, drawings are not necessarily to scale.

While most of the terms used herein will be recognizable to those of ordinary skill in the art, it should be understood that when not explicitly defined, terms should be interpreted as adopting a meaning presently accepted by those of ordinary skill in the art.

The present disclosure provides processes for the recovery of olefins from a mixture. In various embodiments, such processes include: (1) passing the mixture to a first surface of a porous membrane in a first contactor; (2) passing a complexing composition that includes a metal ion capable of reversibly binding the olefins to a second surface of the porous membrane in the first contactor; (3) extracting at least a portion of the olefins from the mixture by binding the olefins to the metal ion of the complexing composition, and thereby producing a mixture depleted in olefins and a complexing composition enriched in olefins (i.e., “olefin-enriched complexing composition”); and (4) inducing a release of the olefins from the olefin-enriched complexing composition.

As set forth below, the processes of the present disclosure have numerous embodiments. For instance, in some embodiments, the induction step may occur in a second contactor. In such embodiments, the processes of the present disclosure further include passing the olefin-enriched complexing composition to a second contactor for the induction step to occur. Likewise, in additional embodiments, the processes of the present disclosure may further include passing the released olefins from the induction step to a third contactor, such as, for example, a collection vessel. In other embodiments, the processes of the present disclosure may further include recycling the complexing composition after the olefins are released in the induction step.

The processes of the present disclosure may be applied to various mixtures. For instance, in some embodiments, the mixtures can be multi-component mixtures, such as mixtures comprising saturated and unsaturated hydrocarbons. Such mixtures may also be olefin/paraffin mixtures. The mixtures may also contain olefinic and non-olefinic hydrocarbons.

The mixtures may also be in various states. For instance, in some embodiments, the mixtures may be in a gaseous phase. In other embodiments, the mixtures may be in a liquid phase. In additional embodiments, the mixtures may be in a liquid and a gaseous phase. In some embodiments, the mixture is normally a gas at standard temperature and pressure but is a liquid at the operating temperatures and pressures of the processes. In some embodiments, the mixture is normally a liquid at standard temperature and pressure but is a gas at the operating temperatures and pressures of the processes.

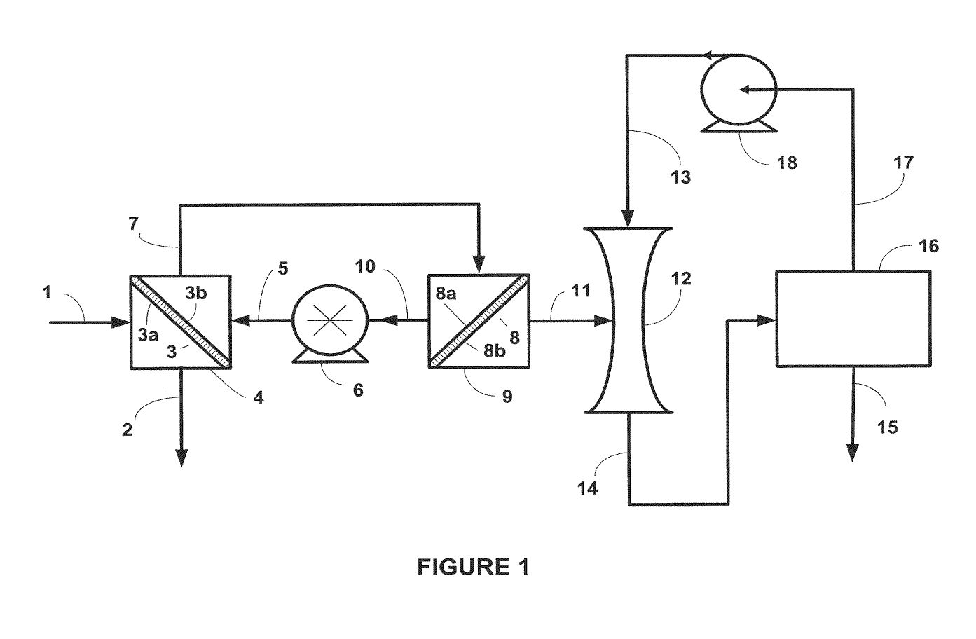

Referring now to FIG. 1, a schematic is shown to illustrate an example of how the olefin recovery processes of the present disclosure may be carried out in some embodiments. In such embodiments, a mixture with olefins is passed through line 1 to first surface 3a of porous membrane 3 in first contactor 4. Likewise, a complexing composition is passed to second surface 3b of porous membrane 3 through line 5 via pump 6. Thereafter, at least a portion of the olefins from the mixture are extracted. In this embodiment, it is envisioned that such extraction occurs by a migration of the olefins in the mixture from first surface 3a of porous membrane 3 to second surface 3a. Thereafter, the olefins bind to the metal ions of the complexing composition. In some embodiments, the binding occurs within porous membrane 3. In other embodiments, the binding occurs on second surface 3a. This produces a mixture depleted in olefins, and a complexing composition enriched in olefins. Next, the olefin-enriched complexing composition is passed through line 7 to second contactor 9 for the induction step.

In the embodiments illustrated in FIG. 1, second contactor 9 includes second porous membrane 8, a membrane selectively permeable to olefins. Second porous membrane 8 also includes first surface 8a and second surface 8b. In addition, second contactor 9 is in association with Venturi nozzle 12 via line 11. In some embodiments, the induction step to release the olefins from the complexing composition occurs by increasing the temperature of second contactor 9 to about 75° C., and decreasing the pressure of the second contactor to about 150 kPa. The temperature of second contactor 9 may be increased by a heating unit (not shown). Likewise, the pressure of second contactor 9 may be decreased by Venturi nozzle 12. In particular, a vacuum on the side of second surface 8b of membrane 8 can be achieved by pumping a liquid motive fluid using pump 18 through line 13 into Venturi nozzle 12. It is envisioned that reduced pressure caused by Venturi nozzle 12 then decomplexes the olefins from the composition complex and pulls the released olefins across membrane 8. Other elements of the composition complex are left behind on the side of first surface 8a. Thereafter, it is envisioned that the olefin product exits as a vapor through line 11 and enters the neck of Venturi nozzle 12. It is also envisioned that the liquid motive fluid from pump 18 condenses olefins to the liquid phase. Thereafter, the liquid olefins along with the liquid motive fluid exit through line 14 into collection vessel 16. The isolated olefins may then be collected from collection vessel 16 through line 15. For normally gaseous olefins, such as, for example, ethylene and propylene, the stream passing through line 14 may also be in two phases (vapor/liquid).

After the olefins become released from the complexing composition, the complexing composition in second contactor 9 may be recycled by passing the complexing composition to first contactor 4 via lines 10 and 5. Such a recycling step can also be facilitated by pump 6. The aforementioned process may then be repeated.

Applicant notes that the aforementioned embodiment discussed above and illustrated in FIG. 1 provides a non-limiting example of how to practice the olefin recovery processes of the present disclosure. In other embodiments, the olefin recovery processes of the present disclosure may be practiced in different manners and variations. In particular, the detailed disclosure below sets forth some of the different ways by which a person of ordinary skill in the art may practice each of the steps of the olefin recovery processes of the present disclosure.

Passing Mixtures and Complexing Compositions to a First Contactor for Extraction

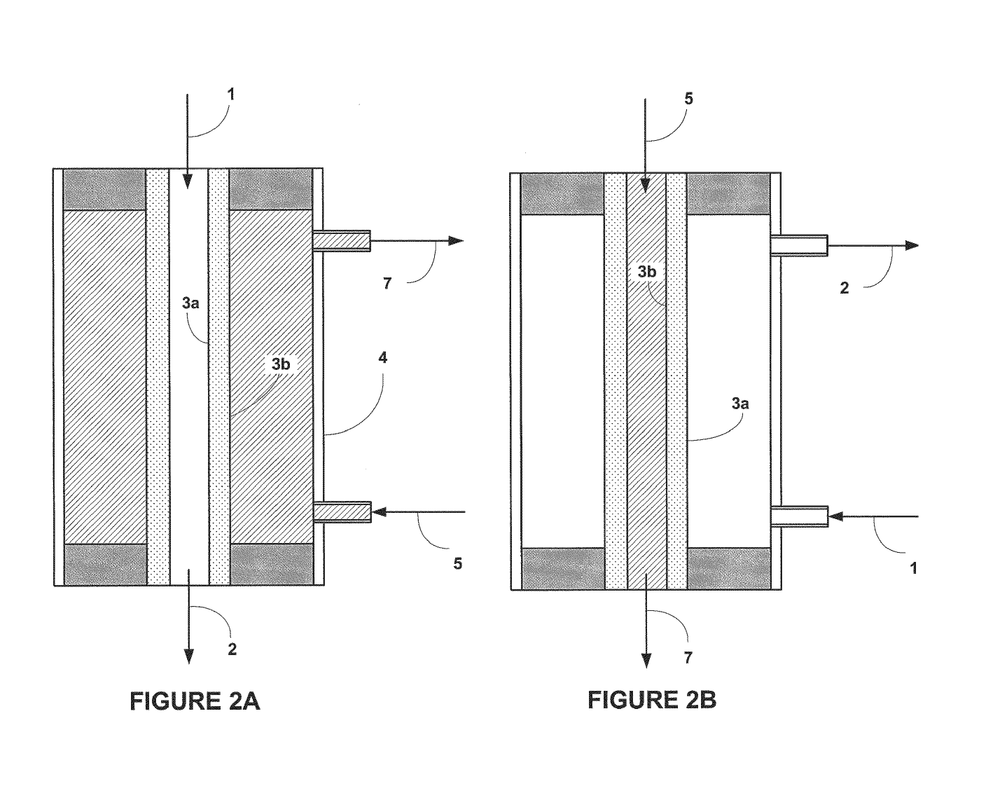

In the present disclosure, extraction occurs in a first contactor. In some embodiments of the present disclosure, the first contactor may be a container, such as container 4 shown in FIG. 1. The first contactors of the present disclosure have a porous membrane with two surfaces. For instance, contactor 4 shown in FIG. 1 has a porous membrane 3 with first surface 3a and second surface 3b. In some embodiments, such porous membranes may contain a plurality of hollow fibers. More specific and non-liming depictions of first contactors suitable for use with the processes of the present disclosure are shown in FIGS. 2A and 2B and will be described in more detail below. However, Applicant notes that the aforementioned first contactors are only specific and non-limiting examples of first contactors that may be used in the present disclosure. Thus, a person of ordinary skill in the art can envision additional suitable first contactors that fall within the scope of the present disclosure that were not disclosed here.

In operation, mixtures with olefins are passed to a first surface of a porous membrane in a first contactor. Likewise, complexing compositions with a metal ion capable of reversibly binding the olefins are passed to a second surface of the porous membrane in the first contactor. Thereafter, olefins are extracted from the mixture by the complexing composition.

Mixtures and complexing compositions may be passed to first contactors of the present disclosure by various methods. For instance, in some embodiment, the passing includes pumping the mixture and the complexing compositions into the first contactor at a fixed rate. In other embodiments, the pumping rate of each component is variable. However, other methods well known in the other may also be used in additional embodiments.

The manners of operation of the first contactors of the present disclosure can be better understood by referring again to FIGS. 2A and 2B. FIG. 2A illustrates one mode of operation claimed in the present disclosure, wherein a mixture is directed through line 1 to the inside

(lumen) of a plurality of hollow fibers and exits through line 2. During passage through the hollow fiber, olefins are extracted by the metal ion in the complexing composition that enters the first contactor through line 5. In particular, it is envisioned that the olefins in the mixture pass through first surface 3a, diffuse through the fiber wall of the porous membrane, pass through second surface 3b and are carried by the complexing composition out of the first contactor through line 7. Therefore, the mixture stream leaving through line 2 can become depleted in olefins. Likewise, the complexing composition stream leaving through line 7 can become enriched in olefins.

FIG. 2B illustrates an alternative and non-limiting mode of operation of the first contactors of the present disclosure, wherein the mixture feed is directed through line 1 to the outside (shell side) of a plurality of hollow fibers of the porous membrane and exits through line 2. During passage around the hollow fibers of the porous membrane, it is envisioned that olefins are extracted by the metal ions of the complexing composition that enter the lumen side of the first contactor through line 5. In particular, it is envisioned that the olefins in the mixture pass through first surface 3a, diffuse through the fiber wall of the porous membrane, pass through second surface 3b, and are carried by the complexing composition out of the first contactor through line 7. Therefore, the mixture stream leaving through line 2 can become depleted in olefins. Likewise, the complexing composition stream leaving through line 7 can become enriched in olefins.

Applicant notes that the operation of first contactors depicted in FIGS. 2A and 2B merely illustrate non-limiting examples of how mixtures and complexing compositions can pass through a first contactor for extraction to occur. Thus, a person of ordinary skill in the art can envision additional suitable modes of operating first contactors that fall within the scope of the present disclosure that were not disclosed here. It can also be appreciated by those skilled in the art that any number of first contactors can be arranged in series or parallel to achieve the desired transport of mixtures and complexing compositions.

Extraction of Olefins from the Mixture

In general, and as mentioned previously, the extraction of olefins from a mixture occurs by binding the olefins to the metal ion of the complexing composition such that the mixture becomes depleted in olefins and the complexing composition becomes enriched in olefins. For instance, in some embodiments, the extraction step may deplete a mixture of about 0.1% to about 95% by weight of its olefins. In more specific embodiments, the extraction step may deplete a mixture of about 0.1% to about 90% by weight of its olefins. In further embodiments, the extraction step may deplete a mixture of about 1% to about 85% by weight of its olefins.

The extraction step may occur by various methods. For instance, as previously shown and described in FIGS. 1, 2A and 2B, extraction can occur by the migration of the olefins in the mixture from the first surface of a porous membrane in a first contactor to the second surface of the porous membrane. Thereafter, the olefins bind to the metal ion of the complexing composition.

The extraction step can also occur in various states. For instance, in some embodiments, extraction may occur when both the mixture and the complexing composition are in liquid phases (hereinafter “liquid-liquid extraction”). In other embodiments, extraction may occur when the mixture is in a gaseous phase and the complexing composition is in a liquid phase (hereinafter “gaseous-liquid extraction”). In additional embodiments, extraction may occur when the mixture is both in a gaseous phase and a liquid phase while the complexing composition is in a liquid phase (hereinafter “liquid/gaseous-liquid extraction”). However, Applicant notes that the aforementioned extraction states are non-limiting examples of how extraction can occur in the olefin recovery processes of the present disclosure. Thus, a person of ordinary skill in the art can envision additional suitable extraction states that fall within the scope of the present disclosure that were not disclosed here.

Without being bound by theory, it is envisioned that extraction occurs because the mixture and complexing composition are immiscible, thereby resulting in the formation of an interface between the mixture and the complexing composition within in the pores of the porous membrane in the first contactor. It is also envisioned that contact of the mixture and the complexing composition at such an interface causes olefins to be selectively extracted from the mixture into the metal ions in the complexing composition.

Binding of olefins to metals ions in a complexing composition can be sensitive to temperature and pressure. In particular, lower temperatures generally favor a greater degree of olefin-metal ion complex formation and higher temperatures generally favor less olefin-metal ion complex formation. Likewise, high pressure generally favors more olefin-metal ion complex formation and lower pressure generally favors less olefin-metal ion complex formation.

Therefore, to obtain optimal results during extraction, the first contactors of the present disclosure are preferably operated at low temperature ranges that favor olefin-metal ion complex formation. For instance, in some embodiments, such temperature ranges may be from about 0° C. to about 50° C. In some preferred embodiments, the first contactor is operated at a temperature range from about 20° C. to about 40° C. In more preferred embodiments, the first contactor is operated at a temperature range from about 25° C. to about 35° C.

The optimal pressure ranges for extracting olefins in the first contactors of the present disclosure can vary with the physical state of the mixtures to be processed. For instance, in some embodiments where the mixture is in a liquid phase, the first contactor may be operated at a pressure range from about 100 kPa to about 300 kPa. In some preferred embodiments, the first contactor is operated at a pressure range from about 110 kPa to about 250 kPa. In more preferred embodiments, the first contactor is operated at a pressure range from about 110 kPa to about 200 kPa. Likewise, in other embodiments where the mixture is in a gaseous phase, the first contactor can be operated at a pressure range from about 100 kPa to about 3,500 kPa. In some preferred embodiments, the first contactor may be operated at a pressure range from about 100 kPa to about 2,000 kPa. In more preferred embodiments, the first contactor is operated at a pressure range from about 200 kPa to about 1,000 kPa.

The ratio of complexing composition to mixture can also be varied in the extraction processes of the present disclosure. In particular, in various embodiments of the present disclosure, the ratio is high enough to capture the olefins within a reasonable residence time but low enough not to cause a significant increase in the process cost. For the processes of the instant disclosure, the preferred range of complexing composition to mixture ratios in volume may be without limitation about 1:1 to about 20:1, more preferably about 2:1 to about 10:1, and most preferably about 3:1 to about 6:1.

Induction of the Release of Olefins from the Complexing Composition

In the present disclosure, the induction of the release of the olefins from an olefin-enriched complexing composition can occur by various methods. For instance, in some embodiments, the induction step includes increasing the temperature of the olefin-enriched complexing composition environment. In other embodiments, the induction step includes decreasing the pressure of the environment. In additional embodiments, the induction step includes increasing the temperature and decreasing the pressure of the environment.

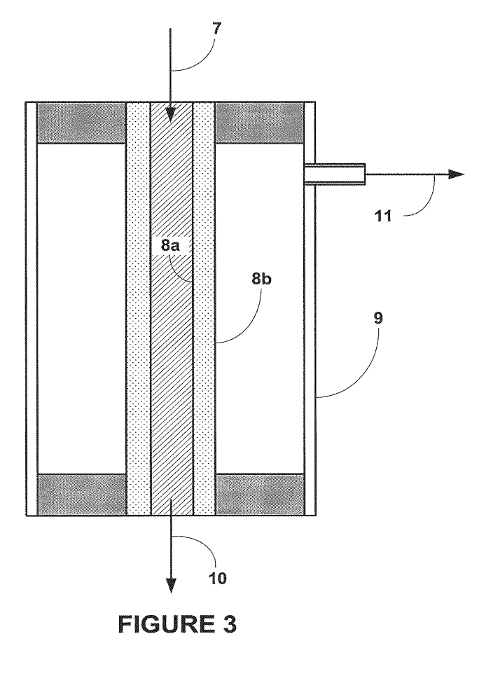

Various embodiments of the present disclosure may utilize a second contactor as the environment to carry out the induction step. Such second contactors may be containers, such as container 9 shown in FIG. 1. Such second contactors may also include a second porous membrane with two surfaces, such as second porous membrane 8 shown in FIG. 1 with first surface 8a and second surface 8b. In this example, porous membrane 8 also includes a plurality of hollow fibers. A more detailed illustration of second contactor 9 is shown in FIG. 3. However, Applicant notes that the aforementioned second contactors are only specific and non-limiting examples of second contactors that may be used in the present disclosure. Thus, a person of ordinary skill in the art can envision additional suitable second contactors that fall within the scope of the present disclosure that were not disclosed here.

In the embodiments that utilize a second contactor, the induction step may also occur by increasing the temperature of the second contactor. For instance, in specific embodiments, the temperature in the second contactor may be increased to less than about 75° C., less than about 60° C., or less than about 50° C. Such increases in temperature may occur by various methods, such as the utilization of heating units, heat exchangers, and the like.

In the embodiments that utilize a second contactor, the induction step may also occur by decreasing the pressure of the second contactor. For instance, in some embodiments, the pressure in the second contactor may be decreased to less than about 75 kPa. In more specific embodiments, decreasing the pressure of the second contactor may be accomplished by the use of a vacuum pump that is in association with the second contactor. In other embodiments, decreasing the pressure of the second contactor may be accomplished by the use of a Venturi nozzle that is in association with the second contactor. An illustration of a Venturi nozzle used for such purposes is shown in FIG. 1 as object 12. In additional embodiments that utilize a second contactor, the induction step may also occur by increasing the temperature and decreasing the pressure of the second contactor.

Referring now to FIGS. 1 and 3, the operation of second contactor 9 will be described in detail as a non-limiting example of an induction step that can be suitable for some embodiments of the olefin recovery processes of the present disclosure. In operation, the complexing composition carrying olefins is directed through line 7 into the inside (lumen side) of second contactor 9 (shown in FIG. 3). As the olefin-enriched complexing composition passes through the length of the hollow fibers of porous membrane 8, a vacuum on the side of second surface 8b of membrane 8 can be achieved by pumping a liquid motive fluid using pump 18 through line 13 into Venturi (converging-diverging) nozzle 12. Thereafter, the vacuum creates a partial pressure driving force for the olefins to desorb from the complexing composition, pass through membrane surfaces 8a and 8b, and be drawn into the system through line 11. Without being bound by theory, it is envisioned that a liquid-vapor interface is also set up in membrane 8 when one utilizes a Venturi nozzle.

In some embodiments, the motive fluid is the same as the solvent of the complexing composition. In other embodiments, the motive fluid is a different high-boiling solvent than that present in the complexing composition. In the case of the present embodiments, the motive fluid is chosen as a high boiling fluid identical to that used in the complexing composition solvent (as discussed below in more detail). For instance, a preferred motive fluid is tetraethylene glycol (TEG). In addition, it is envisioned that liquid motive fluid from pump 18 can function to condense olefins that are liquid at atmospheric pressure, possibly due to the transfer of the heat of condensation to the liquid motive fluid. Furthermore, since the pressure is typically atmospheric in line 14, the olefin condenses to the liquid phase. Thereafter, liquid olefins and liquid motive fluid exit through line 14.

The use of Venturi nozzles for the olefin recovery processes of the present disclosure can have several advantages. For instance, and without being bound by theory, it is envisioned that, because of the high boiling nature of the complexing composition, olefins can be selectively drawn into the vacuum, thereby eliminating any post-treatment for removal of solvent from the desired olefin product. In addition, unlike a vacuum pump, the vacuum achievable with a Venturi nozzle is continuously variable and depends only on the velocity and vapor pressure of the motive fluid.

Without again being bound by theory, the operation of a Venturi nozzle can be better understood by Bernoulli's Law. In particular, it is envisioned that the pressure at the smallest diameter of the Venturi nozzle is related to the pressure at the entrance to the nozzle as well as the fluid velocities as shown below:

P₁=P₂−½ρ(ν₁²−ν₂²)

where, P refers to pressure, p refers to the working fluid density, v refers to the fluid velocity, subscript 1 refers to the reduced diameter (“neck”) of the Venturi nozzle where the vacuum is created, and subscript 2 refers to the diameter of the upstream region of the neck.

Collection of Olefins

In additional embodiments, the processes of the present disclosure may further include passing the released olefins from the induction step to a third contactor, such as a collection vessel. For instance, as shown in FIG. 1, collection vessel 16 can be used in one embodiment as a simple decanter vessel. In such embodiments, liquid olefins and liquid motive fluid that exit through line 14 can enter collection vessel 16. Other well-known third contactors may also be suitable for use with the processes of the present disclosure.

The use of collection vessels may be desirable in some embodiments of the present disclosure where neither liquid olefins nor gaseous olefins may be soluble in a particular motive fluid. Therefore, in such embodiments, the olefin can separate into a second phase in the collection vessel and be readily recovered with minimal additional heat consumption. Furthermore, and as depicted in FIG. 1, the motive fluid in such embodiments may then exit collection vessel 16 through line 17 and become recycled to the Venturi nozzle 12 through line 13 by pump 18.

Recycling of the Complexing Composition

In some embodiments of the present disclosure, the olefin recovery processes may further include recycling the complexing composition after the olefins are released in the induction step. Such a recycling step may include passing the complexing composition to the second surface of the porous membrane in the first contactor after the release of the olefin from the complexing composition. For instance, as shown in FIG. 1, the complexing composition in second contactor 9 may be recycled by passing the complexing composition to first contactor 4 via lines 10 and 5. Such a recycling step can also be facilitated by pump 6. The olefin recovery processes may then be repeated. Furthermore, and as set forth below, numerous complexing compositions are suitable for use with the olefin recovery processes of the present disclosure.

Complexing Compositions

The complexing compositions of the present disclosure generally include a metal ion capable of reversibly binding the olefins. Such complexing compositions can have numerous embodiments. For instance, Applicant's pending patent application No. PCT/US09/46559, the entirety of which is incorporated herein by reference, includes numerous examples of complexing compositions that are suitable for use in the olefin recovery processes of the present disclosure.

In some non-limiting examples, the metal ion in a complexing composition is a transition metal ion, such as Cu⁺. In more specific embodiments, the complexing composition may further include a counter anion, a ligand, and a solvent. In more preferred embodiments, the complexing compositions of the present disclosure may include: (1) a transition metal ion; (2) a counter anion; (3) a ligand selected from the group consisting of a bidentate ligand and a tridentate ligand, wherein the ligand includes at least two nitrogen atoms, and wherein each of the nitrogen atoms includes a lone pair of electrons; and (4) a polar solvent with a boiling point of at least about 200° C.

A detailed description of each of the aforementioned complexing composition components is included below. However, Applicant notes that such a description represents non-limiting examples of complexing composition components that may be used in the present disclosure. Thus, a person of ordinary skill in the art can envision additional suitable complexing compositions with different components that fall within the scope of the present disclosure that were not disclosed here.

Transition Metal Ions.

In some embodiments, the transition metal ion of the complexing compositions of the present disclosure is Cu⁺. Such a Cu⁺ ion in the present disclosure may be obtained in a number of non-limiting ways. For instance, the Cu⁺ ion may be obtained from cuprous salts, such as CuCl, CuI, CuBr or CuCN. However, though such salts are readily available, they may not always be soluble in a solvent of choice for various embodiments of the present disclosure. Therefore, in other embodiments, Cu⁺ coordination complexes with acetonitrile may be purchased commercially for use as a transition metal ion. Such complexes usually consist of Cu⁺ ions coordinated in all four available positions with acetonitrile and a fixed anion such as the hexafluorophosphate ion (PF₆⁻¹). This material is referred to as tetrakis(acetonitrile)copper(I) hexafluorophosphate. In solution, the monodentate acetonitrile ligands are easily exchanged for more stable bidentate or tridentate ligands.

In other embodiments, Cu⁺ may be made in-situ by reducing a Cu⁺⁺ salt such as Cu(NO₃)₂.2.5H₂O using elemental copper (Cu⁰) in acetonitrile to form tetrakis(acetonitrile)copper(I) nitrate. However, it should be recognized that anyone skilled in the art may select other salts that may produce an acceptable Cu(I) coordination complex with any number of ligands.

In other embodiments, the transition metal ion of the complexing compositions of the present disclosure is Ag⁺. Such Ag⁺ ions may also be obtained in a number of non-limiting ways, as are known by persons of ordinary skill in the art.

Furthermore, Applicant notes that the aforementioned transition metal ions are only specific and non-limiting examples of transition metal ions that may be used in the present disclosure. Thus, a person of ordinary skill in the art can also envision additional suitable transition metal ions that fall within the scope of the present disclosure that were not disclosed here.

Counter Anions

In some embodiments, counter anions that are suitable for use in the complexing compositions of the present disclosure include but are not limited to hexafluorophosphate (PF₆⁻¹), tetrafluoroborate (BF₄⁻¹), nitrate (NO₃⁻¹) and tetraphenylborate (BPh₄⁻¹). In various embodiments, the counter anions of the present disclosure are non-coordinating anions. By way of example, and without being bound by theory, the selection of counter anions in the present disclosure may be based on measurable interactions. For example, tetrafluoroborate has the possibility of a B—P^{. . .} Cu interaction that may compete with the Cu ^{. . .} olefin binding. However, the equivalent interaction for tetraphenylborate (i.e., Ph ^{. . .} Cu) may be weaker.

In further embodiments, counter anions suitable for use in the complexing compositions of the present disclosure may also be simple halides, such as chloride (Cl⁻¹), iodide (I⁻¹), bromide (Br⁻¹) and fluoride (F⁻¹). In further embodiments, counter anions may be carboxylate anions (COO⁻). However, the aforementioned halides and carboxylate anions may also be capable of competing as ligands due to their lone pair of electrons. Accordingly, complexing compositions made using such species may, at least in some embodiments, undergo disproportionation to Cu⁺⁺ and Cu⁰.

In other embodiments, the counter anion is selected from the group consisting of PF₆⁻¹, BF₄⁻¹, NO₃⁻¹, BPh₄⁻¹, Cl⁻¹, I⁻¹, Br⁻¹, F⁻¹, and COO⁻¹. In various embodiments, the counter anion includes a non-coordinating anion.

Applicant also notes that the aforementioned counter anions are only specific and non-limiting examples of counter anions that may be used in the present disclosure. Thus, a person of ordinary skill in the art can envision additional suitable counter anions that fall within the scope of the present disclosure that were not disclosed here.

### Ligands

By way of background, transition metal ions are Lewis acids that form stable Lewis Acid-Base adducts with Lewis bases. Ligands are Lewis bases because they bear at least one atom having a lone pair of electrons. For instance, ligands such as H₂O, NH₃, CO, OH⁻¹, and CN⁻¹ that bear a single Lewis base atom are termed monodentate ligands. Likewise, ligands bearing two such atoms are termed bidentate ligands. Similarly, ligands that bear three Lewis base atoms are termed tridentate ligands.

Monodentate ligands such as pyridine can interact with Cu⁺ to form a copper complex that can be used in the complexing compositions to separate olefins. Such monodentate copper complexes are often unstable, however. Tetradentate ligands, in which the lone pairs are separated by several intervening atoms, can occupy all four dₓ²_{y}² orbitals of a transition metal ion to form stable complexes known as chelates. Such chelate complexes may not have the ability to interact with electrons from an olefin for binding and separation to occur. Likewise, polydentate ligands that contain more than four lone pairs of electrons have the same olefin binding limitations. However, such limitations generally do not apply to bidentate or tridentate ligands.

Accordingly, in some embodiments, ligands suitable for use with the complexing compositions of the present disclosure are selected from the group consisting of bidentate and tridentate ligands. Such bidentate and tridentate ligands desirably include at least two nitrogen atoms, each with a lone pair of electrons. In other embodiments, the bidentate or tridendate ligand may include two or more aromatic rings, where each of the aromatic rings may include at least one nitrogen atom with a lone pair of electrons. In other embodiments, the aromatic rings may be connected to each other by carbon or nitrogen linkages. For instance, a general structure for a ligand suitable for use with the complexing compositions of the present disclosure is shown below as a non-limiting example:

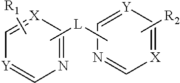

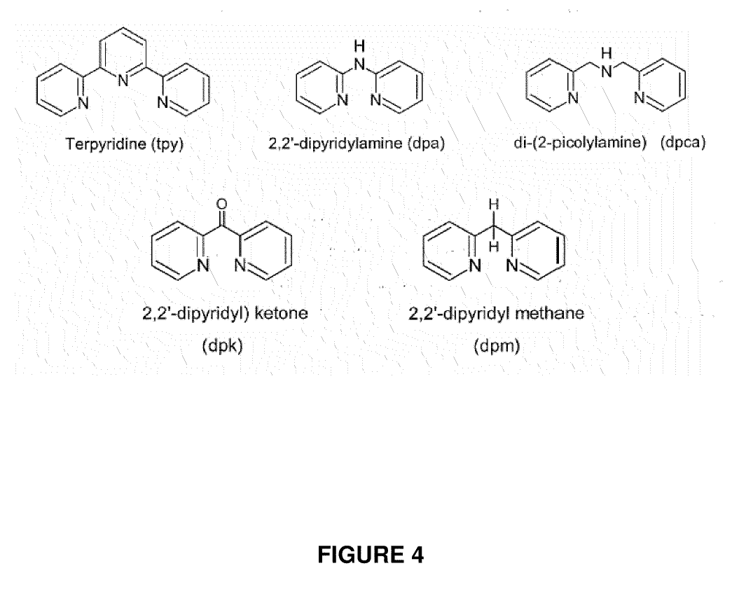

In this generalization, X and Y represent either carbon (C) or nitrogen (N). Likewise, R₁ and R₂ represent substituents on the aromatic rings at any allowable position. Such substituents may be alkyl or aromatic in nature. In addition, L represents a linking group which may include any of the groups shown below:

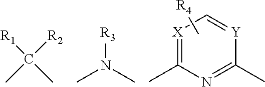

where R₁, R₂, and R₄ represent substituents that may include: (1) a single atom such as H, F, Cl, Br or I; (2) an alkyl group; or (3) an aromatic ring. Likewise, R₃ represents substituents that may include: (1) a single atom such as H; (2) an alkyl group; or (3) an aromatic ring. Non-limiting examples of such ligands are shown in FIG. 4.

A person of ordinary skill in the art will recognize that numerous ligands may be suitable for use with the complexing compositions of the present disclosure. Furthermore, such ligands may have various physical properties. For instance, in some embodiments, the ligand is a bidentate ligand. In additional embodiments, the bidentate ligand has a boiling point of at least about 200° C. In further embodiments, the bidentate ligand has a vapor pressure of less than about 0.01 kPa at 20° C. However, in additional embodiments, the bidentate ligand may have a vapor pressure of less than about 0.005 kPa at 20° C., or less than about 0.001 kPa at 20° C. In more specific embodiments, the bidentate ligand includes at least two aromatic rings, wherein each of the aromatic rings includes a nitrogen atom with a lone pair of electrons. In additional embodiments, the bidentate ligand is selected from the group consisting of 2,2′-dipyridyl amine, 2,2′-dipyridyl ketone and 2,2′-dipyridyl methane.

In other embodiments, the ligand is a tridentate ligand. In additional embodiments, the tridentate ligand has a boiling point of at least about 200° C. In further embodiments, the tridentate ligand has a vapor pressure of less than about 0.01 kPa at 20° C. However, in other embodiments, the tridentate ligand may have a vapor pressure of less than about 0.005 kPa at 20° C., or less than about 0.001 kPa at 20° C. In more specific embodiments, the tridentate ligand includes at least two aromatic rings, wherein each of the aromatic rings includes a nitrogen atom with a lone pair of electrons. In additional embodiments, the tridentate ligand is selected from the group consisting of terpyridine and di-(2-picolylamine).

The chemical structures of exemplary bidentate and tridentate ligands are shown in FIG. 4 as non-limiting examples. However, Applicant notes that the ligands shown in FIG. 4 and described in this specification are only specific and non-limiting examples of ligands that may be used with the complexing compositions of the present disclosure. Thus, a person of ordinary skill in the art can envision additional suitable ligands that fall within the scope of the present disclosure that were not disclosed here.

Solvents

Various solvents may also be used with the complexing compositions of the present disclosure. In some embodiments, the solvent is a high boiling solvent (i.e., a solvent with a high boiling point, such as, for example, a boiling point of at least about 200° C.). In other embodiments, the solvent is a polar solvent with acceptable electronic properties (e.g., dipole moment, polarizability, etc.).

In further embodiments, the solvent may also have low a vapor pressure. For instance, in some embodiments, the solvent has a vapor pressure of less than about 0.01 kPa at 20° C. In other embodiments, the solvent may have a vapor pressure of less than about 0.1 kPa at 20° C., less than about 0.05 kPa at 20° C., or less than about 0.005 kPa at 20° C.

In other embodiments, the solvent may have one or more of the following physical properties: (1) a boiling point greater than about 200° C.; (2) a vapor pressure of less than about 0.005 kPa at 20° C.; and (3) a viscosity lower than 100 mPa·s at 25° C.

In other embodiments, the boiling point of the solvent is higher than the boiling point of the highest boiling olefin in the mixture. For instance, in some embodiments, the boiling point of the solvent is at least about 20° C. higher than the boiling point of the highest boiling olefin in the mixture. In other embodiments, the boiling point of the solvent is at least about 50° C. higher than the boiling point of the highest boiling olefin in the mixture. In still other embodiments, the boiling point of the solvent is at least about 100° C. higher than the boiling point of the highest boiling olefin in the mixture.

A non-limiting example of a solvent suitable for use with the complexing compositions of the present disclosure may be a polyalkylene glycol with the following general formula:

H—(O—CH₂CH₂)ₙ—OH 2≦n≦10.

In various embodiments, n represents a value ranging from 2 to 10. However, in other embodiments, n may have different value ranges. In more specific embodiments, n represents a value ranging from 2 to 6. In other embodiments, the polyalkylene glycol is selected from the group consisting of diethylene glycol, triethylene glycol, tetraethylene glycol, pentaethylene glycol and hexaethylene glycol.

In additional embodiments, the solvent may be adiponitrile. In other embodiments, the solvent includes an ionic liquid. In more specific embodiments, the ionic liquid is selected from the group consisting of 1-butyl-3-methylimidazolium hexafluorophosphate, 1-ethyl-3-methylimidazolium tetrachloroaluminate, 1-butylpyridinium nitrate, 1-butyl-3-methylimidazolium tetrafluoroborate and mixtures thereof.

Applicant also notes that the aforementioned solvents are only specific and non-limiting examples of solvents that may be used with the complexing compositions of the present disclosure. Thus, a person of ordinary skill in the art can envision additional suitable solvents that fall within the scope of the present disclosure that were not disclosed here.

Analysis

In summary, the olefin recovery processes of the present disclosure are useful for the separation of olefins from various mixtures. In selecting complexing compositions for use with the processes of the present disclosure, one must consider the various attributes of the components of such complexing compositions. For instance, one attribute is that Ag⁺ is expensive and generally unstable. A second attribute is that Ag⁺ and Cu⁺ transition metals can have significant effects on the behavior of the complexing compositions toward olefins.

A third attribute is that the use of a solvent or ligand with a high vapor pressure (e.g., higher than about 100 kPa) may affect the olefin separation process. For instance, when such solvents are used in a gas phase absorption process (such as separation of ethylene from ethane or propylene from propane), a portion of that solvent or ligand may become volatilized into the non-absorbed gas stream, thus requiring an additional and costly separation step downstream.

A fourth attribute is that, water, while acceptable as a solvent for Ag⁺ ions, is known to promote the disproportionation of Cu⁺ into Cu⁺⁺ and Cu⁰ if the copper is not adequately coordinated by a ligand. Thus, Cu⁺ may not be suitable for all complexing compositions of the present disclosure.

Finally, a fifth attribute is that monodentate nitrogen ligands (like pyridine) are not as effective in stabilizing Cu⁺ as are bidentate or tridentate ligands. Without being bound by theory, it is envisioned that such different stabilities may be based on the principle that the stability of the metal-ligand complexes increase in the following order: monodentate<bidentate<tridentate<tetradentate. Monodentate ligands are generally reversible and tend to have lower boiling points. Therefore, they may not be optimal for use in various embodiments of the present disclosure. On the other hand, tetradentate ligands stably occupy all coordination sites leaving no room for the olefin. Therefore, the preferred ligands for the complexing compositions of the present disclosure are bidentate and tridentate ligands.

Finally, one must also keep in mind that, in the absence of a suitable ligand to stabilize it, Cu⁺ will disproportionate into Cu⁺⁺ and Cu⁰, neither of which is capable of binding olefins. Further, a metal ion stabilized by a ligand has been shown to more efficiently complex olefins if it is dissolved in a suitable solvent.

The above attributes and factors were considered in devising the complexing compositions of the present disclosure for the olefin recovery processes of the present disclosure. However, based on Applicant's current awareness, such attributes and factors were not considered in the prior art. In addition, Applicant is currently unaware of any similar complexing compositions or olefin recovery processes in the prior art.

From the foregoing description, one skilled in the art can easily ascertain the essential characteristics of this disclosure, and without departing from the spirit and scope thereof, can make various changes and modifications to adapt the disclosure to various usages and conditions. Therefore, the embodiments described hereinabove are meant to be illustrative only and should not be taken as limiting of the scope of the disclosure, which is defined in the following claims.

What is claimed is:

1. A process for the recovery of olefins from a mixture, wherein the process comprises:
passing the mixture to a first surface of a porous membrane, wherein the porous membrane is in a first contactor;
passing a complexing composition to a second surface of the porous membrane in the first contactor, wherein the complexing composition comprises a metal ion that is capable of reversibly binding the olefins;
extracting at least a portion of the olefins from the mixture;
wherein extracting comprises binding the olefins to the metal ion of the complexing composition; and
wherein extracting produces a mixture depleted in olefins and a complexing composition enriched in olefins; and
inducing a release of the olefins from the complexing composition enriched in olefins.

2. The process of claim 1, further comprising passing the complexing composition enriched in olefins to a second contactor; and wherein the induction step occurs in the second contactor.

3. The process of claim 2, wherein the induction step comprises increasing the temperature of the second contactor.

4. The process of claim 2, wherein the induction step comprises decreasing the pressure of the second contactor.

5. The process of claim 2, wherein the induction step comprises increasing the temperature and decreasing the pressure of the second contactor.

6. The process of claim 4, wherein decreasing the pressure is accomplished with a Venturi nozzle, and wherein the Venturi nozzle is in association with the second contactor.

7. The process of claim 4, wherein the pressure in the second contactor is decreased to less than about 150 kPa.

8. The process of claim 3, wherein the temperature in the second contactor is increased to less than about 75° C.

9. The process of claim 2, wherein the second contactor further comprises a second porous membrane that is permeable to the olefins, and wherein the released olefins become substantially separated from the complexing composition by passing through the second porous membrane.

10. The process of claim 1, further comprising passing the released olefins from the induction step to a third contactor.

11. The process of claim 1, further comprising recycling the complexing composition after the olefins are released in the induction step.

12. The process of claim 1, wherein the mixture is in a gaseous phase.

13. The process of claim 1, wherein the mixture is in a liquid phase.

14. The process of claim 1, wherein the porous membrane comprises a plurality of hollow fibers.

15. The process of claim 1, wherein the first contactor is operated at a temperature range from about 0° C. to about 50° C.

16. The process of claim 1, wherein the mixture is in a liquid phase, and wherein the first contactor is operated at a pressure range from about 100 kPa to about 300 kPa.

17. The process of claim 1, wherein the mixture is in a gaseous phase, and wherein the first contactor is operated at a pressure range from about 100 kPa to about 3,500 kPa.

18. The process of claim 1, wherein the metal ion is a transition metal ion.

19. The process of claim 1, wherein the metal ion is a transition metal ion, and wherein the complexing composition further comprises:
a. a counter anion;
b. a ligand selected from the group consisting of a bidentate ligand and a tridentate ligand, wherein the ligand comprises at least two nitrogen atoms, and wherein each of the nitrogen atoms comprises a lone pair of electrons; and
c. a polar solvent with a boiling point of at least about 200° C.

20. The process of claim 19, wherein the transition metal ion is Cu⁺.

21. The process of claim 19, wherein the counter anion is selected from the group consisting of PF₆⁻¹ BF₄⁻¹ NO₃⁻¹ BPh₄⁻¹, Cl⁻¹, I⁻¹, Br⁻¹, F⁻¹, and COO⁻.

22. The process of claim 19, wherein the ligand is a bidentate ligand selected from the group consisting of 2,2′-dipyridyl amine, 2,2′-dipyridyl ketone and 2,2′-dipyridyl methane.

23. The process of claim 19, wherein the ligand is a tridentate ligand selected from the group consisting of terpyridine and di-(2-picolylamine).

24. The process of claim 19, wherein the solvent is a polyalkylene glycol.

25. The process of claim 19, wherein the solvent is an ionic liquid.

26. The process of claim 1, wherein the extraction comprises a migration of the olefins in the mixture from the first surface of the porous membrane to the second surface of the porous membrane.

27. The process of claim 1, wherein the extraction comprises a liquid-liquid extraction.

28. The process of claim 1, wherein the extraction comprises a gaseous-liquid extraction.

29. The process of claim 1, wherein the extraction depletes the mixture of about 0.1% to about 95% by weight of the olefins.
